(19) United States
(12) Patent Application Publication
(10) Pub. No.: US 2010/0292660 A1
(43) Pub. Date: Nov. 18, 2010

(54) DERMAL DELIVERY DEVICE WITH ULTRASONIC WELD

(76) Inventor: Agis Kydonieus, Princeton, NJ (US)

Correspondence Address: COOLEY LLP;ATTN: Patent Group, Suite 1100, 777 - 6th Street, NW, WASHINGTON, DC, 20001, US

(21) Appl. No.: 12/668,327

(22) PCT Filed: Jul. 10, 2008
(86) PCT No.: PCT/US08/69622

§ 371 Date: Jun. 4, 2010

(30) Foreign Application Priority Data
Jul. 10, 2007 (US) 60948759

(51) Int. Cl. A61F 13/02; B29C 65/08; A61K 9/70

(52) U.S. Cl. 604/307; 156/73.4

(57) ABSTRACT

A method of enclosing an active ingredient layer between a release liner and a backing layer of a dermal patch by ultrasonically welding a seal between the backing layer and the release liner. A pressure sensitive layer is further provided between the backing layer and release liner.

### CROSS REFERENCE TO RELATED APPLICATION

The present application claims the benefit of U.S. Provisional Application No. 60/948,759, filed on Jul. 10, 2007 and entitled “Dermal Delivery Device with Ultrasonic Weld”, which is herein incorporated by reference in its entirety for all purposes.

### FIELD OF THE INVENTION

This invention is in the field of delivery of pharmacologically or cosmetically active agents to the skin for systemic, local, or topical administration.

### BACKGROUND OF THE INVENTION

A dermal delivery device is an adhesive “patch” for application to the skin that is used to deliver a wide variety of pharmacologically and cosmetically active agents. Such patches can be used to deliver an agent transdermally, i.e., through the skin and into the bloodstream for systemic treatment or into or through the skin for local treatment. Such patches can also be used to administer topical treatments, including cosmetically active agents.

Such patches generally comprise, in addition to the active ingredient, i.e., the pharmaceutically or cosmetically active agent, an adhesive, a backing, and a release liner. In some cases, the device comprises one or more volatile components. Such volatile ingredients can be the active drugs, chemicals to enhance the delivery of the drugs or other excipients important in the development of the proper functioning of the patch, such as solubilizers, humectants and plasticizers.

Such volatile components tend to escape from the device thereby adversely affecting the shelf life of the device and possibly also adversely affecting the effectiveness of the device during use.

### SUMMARY OF THE INVENTION

This invention relates to a method of enclosing an active ingredient layer, especially an active ingredient layer comprising a volatile component, between a release liner and a backing layer, and to dermal patches comprising such sealed active ingredient layer.

### BRIEF DESCRIPTION OF THE FIGURES

FIG. 1 illustrates an exploded cross-section of an illustrative dermal delivery system of the invention.

FIG. 2 illustrates an exploded cross-section of an illustrative dermal delivery system of the invention.

FIG. 3 illustrates an exploded cross-section of an illustrative dermal delivery system of the invention.

FIG. 4 illustrates a plan view of a welded backing layer showing through cuts and restraint tabs.

### DETAILED DESCRIPTION OF THE INVENTION

Following is a description of illustrative embodiments of the invention, with reference to the Figures.

FIG. 1 is a schematic diagram of a patch comprising an active portion, i.e., an active ingredient (AI)-containing patch or layer (5), an internal backing layer (4), a weldable backing layer (or barrier layer) (3), an overlay and a release liner (6). The AI-containing patch comprises the active ingredient and a pressure sensitive adhesive (PSA) as well as excipients used to optimize delivery of the AI from the patch. Layer 4 is an internal backing layer, which provides mechanical support to the AI-containing layer and also prevents direct loss of the actives and other components of the active layer into the overlay. The overlay comprises an overlay covering (1) and a PSA layer (2). Between the overlay and the internal backing layer (4) is an ultrasonically weldable backing layer (3) coated with a tie coat. The tie coat serves to adhere the internal backing layer (4) to the backing layer (3). The internal backing layer also has a tie coat to help adhere the internal backing layer to the AI layer. The release liner (6) comprises a polymer film with good barrier properties such as a polyester or polyacrylonitrile film, which is coated with a release coating such as silicone or fluoropolymer. The release coating prevents strong adhesion between the release liner (6) and the PSA of the overlay (2) or the AI layer. In this drawing an ultrasonically formed hermetic peripheral peelable weld (7) is shown between the release liner (6) and the ultrasonically weldable barrier layer (3). The weld line is proximate to but outside of the perimeter of the AI-containing patch. By proximate is meant within about 1 cm, e.g., about 0.5 cm, about 0.1 cm, about 0.05 cm.

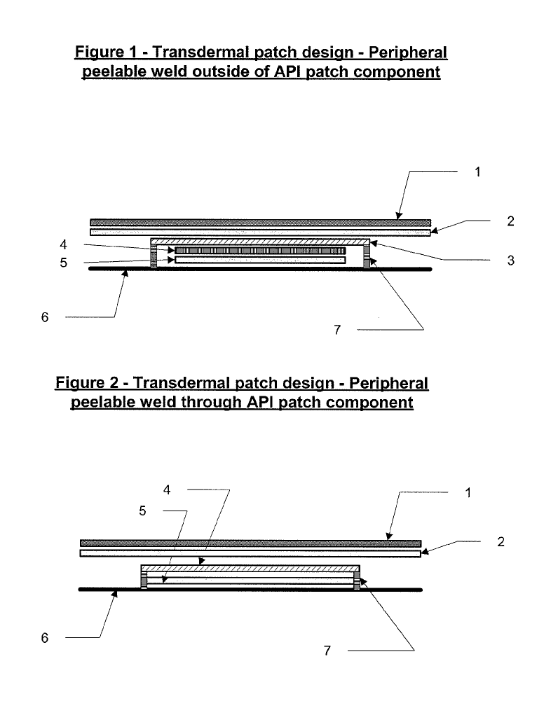

FIG. 2 is a schematic diagram of a patch similar to the one presented in FIG. 1, except that the seal is formed between the internal backing layer (4) and the release liner (6). In this illustrative embodiment, the ultrasonic weld (7) is proximate to and within the perimeter of the AI-containing patch (5) and is formed through: the release coating of the release liner (6), the AI-containing layer (5), and the ethylene vinyl acetate (EVA) tie coat on the internal backing layer (4). This process permits use of a single backing layer (4) and can reduce the overall size of the patch.

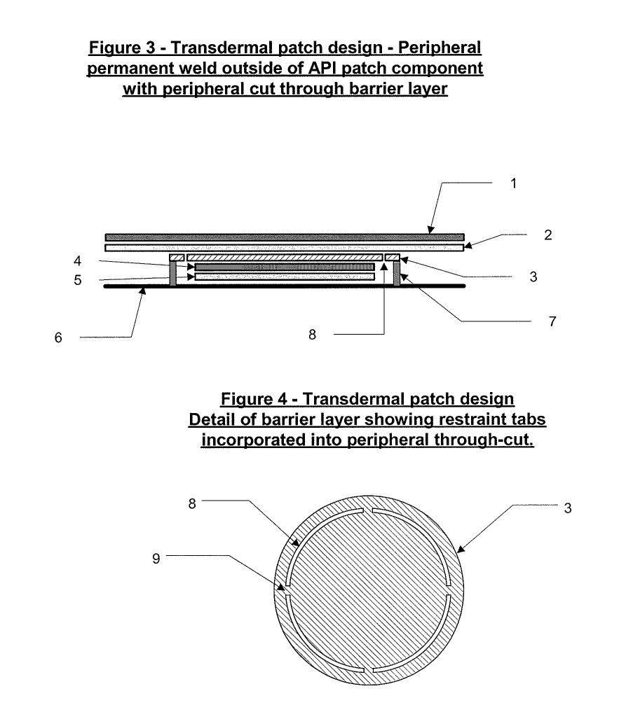

The layers of FIG. 3 are the same as those of FIG. 1, except that the weld (7) between weldable backing layer (3) and release liner (6) is permanent and not peelable and the weldable backing layer (3) is cut through (8). As illustrated, the cut (8) is a “peripheral cut through.” This process is usually performed with a steel rule die, so there is no loss of polymer during the process and consequently an extremely small area through which loss of the volatile excipients can take place. When the release liner is peeled off, a part of the weldable backing layer (3), specifically, the part of the backing layer that is on and outside of the “cut through” line, will also be removed exposing the active portion of the patch and the PSA of the overlay covering. FIG. 4 shows a top view of the weldable backing layer (3) in the embodiment illustrated in FIG. 3. In this illustration the peripheral cut through (8) is intermittent, i.e., the cut is not complete along the entire perimeter, leaving restraint tabs (9) interspersed along the periphery.

In an alternative embodiment, which may be especially useful if the volatile excipients are extremely fugitive, backing layer (3) can be kiss cut. A kiss cut is a cut through only a part of the thickness of the layer and not completely through. The kiss cut can be, e.g., about 50% to about 80% through the layer depending on the requirements. The kiss cut can take place from either side of the weldable backing layer (3), i.e., from the overlay side or from the release liner side. This approach will have better control of extremely fugitive chemicals, as the active portion of the patch is completely encased. The kiss cut would provide a tear-line, which would allow weldable backing layer (3) to separate along the tear line with a pull force that is less than the pull force necessary to separate weldable backing layer (3) from the overlay and that is easily generated by normal finger strength in a healthy adult.

Such design can be used as a child-proof packaging configuration for delivery systems in which the AI-containing layer (5) contains a chemical ingredient (such as Fentanyl) which could prove harmful to small children. The difficulty of tearing through the kiss cut is adjusted such that smaller children would not ordinarily have enough finger strength to expose the AI. This embodiment is useful irrespective of whether the AI-containing layer (5) contains any volatile components.

It should be apparent from the above that alternative patch designs that exploit the inventive contributions herein disclosed can be made by persons skilled in the art. For example, in yet another illustrative embodiment, with references to FIGS. 1 and 3, the overlay covering (1) is ultrasonically welded to the release liner (6), thereby making it unnecessary to include weldable backing layer (3). In this alternative, the weld is formed through the PSA layer (2) and through the release coating on the release liner (6).

The AI in the AI-containing layer can be, for example, one or more active pharmaceutical ingredients (API). In an illustrative embodiment, the API is one or more hormones such as a progestin, e.g., levonogestrel, and an estrogen, e.g., ethinyl estradiol or 17-β estradiol, dispersed in an adhesive polymer matrix. In another aspect of the invention for delivery of a hormone, the API is limited only to a progestin. In other such aspects, the API comprises a progestin, an estrogen, or a testosterone, alone or in any combination thereof.

Other APIs that can also be delivered in accordance with this invention include “small molecules”, i.e., low molecular weight (e.g., <2000 Daltons) synthetic organic compounds such as but not limited to fentanyl, nicotine, scopolamine, nitroglycerine, clonidine, methylphenidate, lidocaine, prilocaine, oxybutynin, antipsychotics such as fluphenazine, alprazolam, risperidone, and olanzapine, Parkinsons drugs such as rotigotine and selegilene Alzheimer's drugs such as rivastigmine and donepezil, anti-hypertensives such as enalapril, BPH drugs such as tamsulosin and terazosin, and anti-asthma drugs such as albuterol and montelukast.

The AI can also be a cosmetic agent such as keratolytic agents such as alpha- and beta-hydroxycarboxylic acids and beta-ketocarboxylic acids; alpha-hydroxy acids such as glycolic acid, lactic acid, tartaric acid, malic acid, citric acid, mandelic acid and, in general, fruit acids; beta-hydroxy acids such as salicylic acid and its derivatives; antibacterials such as clindamicyn or erythromycin phosphate, or antibiotics of the tetracycline type; ascorbic acid and its biologically compatible salts and esters; enzymes; tautening agents such as protein, soya and wheat powders; hydroxylated polyacids; sucroses and their derivatives; urea; amino acids; plant and yeast extracts; protein hydrolysates such as collagen and elastin hydrolysates; hyaluronic acid; mucopolysaccharides; vitamins; panthenol; folic acid; acetylsalicylic acid; allantoin; kojic acid; hydroquinone; retinoic acid and derivatives thereof; fatty acids; etc.

The AI-containing layer can be a polymer matrix comprising the pharmaceutically or cosmetically active ingredient. The polymer can be a PSA to form a biologically acceptable adhesive polymer matrix, preferably capable of forming thin films or coatings through which the AI can pass at a controlled rate. Suitable polymers are biologically and pharmaceutically compatible, nonallergenic, insoluble in and compatible with body fluids or tissues with which the device is contacted. The use of water soluble polymers is generally less preferred since dissolution or erosion of the matrix would affect the release rate of the AI as well as the capability of the dosage unit to remain in place on the skin. So, in certain embodiments, the polymer is non-water soluble.

Preferably, polymers used to form a polymer matrix in the AI-containing layer have glass transition temperatures below room temperature. The polymers are preferably non-crystalline but may have some crystallinity if necessary for the development of other desired properties. Cross-linking monomeric units or sites can be incorporated into such polymers. For example, cross-linking monomers that can be incorporated into polyacrylate polymers include polymethacrylic esters of polyols such as butylene diacrylate and dimethacrylate, trimethylol propane trimethacrylate and the like. Other monomers that provide such sites include allyl acrylate, allyl methacrylate, diallyl maleate and the like.

A useful adhesive polymer formulation comprises a polyacrylate adhesive polymer of the general formula (I):

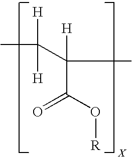

wherein X represents the number of repeating units sufficient to provide the desired properties in the adhesive polymer and R is H or a lower (C₁-C₁₀) alkyl, such as ethyl, butyl, 2-ethylhexyl, octyl, decyl and the like. More specifically, it is preferred that the adhesive polymer matrix comprises a polyacrylate adhesive copolymer having a 2-ethylhexyl acrylate monomer and approximately 50-60% w/w of vinyl acetate as a co-monomer. An example of a suitable polyacrylate adhesive copolymer for use in the present invention includes, but is not limited to, that sold under the tradename of Duro Tak® 87-4098 by National Starch and Chemical Co., Bridgewater, N.J., which comprises a certain percentage of vinyl acetate co-monomer.

The polymeric matrix can also comprise excipients such as skin permeation enhancers, solubilizers, plasticizers, humectants, and tackifiers. A plasticizer/humectant can be dispersed within the adhesive polymer formulation. Incorporation of a humectant in the formulation allows the dosage unit to absorb moisture on the surface of skin which in turn helps to reduce skin irritation and to prevent the adhesive polymer matrix of the delivery system from failing. The plasticizer/humectant may be a conventional plasticizer used in the pharmaceutical industry, for example, polyvinyl pyrrolidone (PVP). In particular, PVP/vinyl acetate co-polymers, such as those having a molecular weight of from about 50,000, are suitable. The PVP/vinyl acetate acts as both a plasticizer, acting to control the rigidity of the polymer matrix, as well as a humectant, acting to regulate moisture content of the matrix. The PVP/VA can be, for example, PVP/VA S-630 which is a 60:40::PVP:VA co-polymer that has a molecular weight of 51,000 and a glass transition temperature of 110° C. The amount of humectant/plasticizer is directly related to the duration of adhesion of the overlay. The PVP/vinyl acetate can be PVP/VA S-630 supplied by International Specialty Products, Inc. (ISP) of Wayne, N.J., wherein the PVP and the vinyl acetate are each present in approximately equal weight percent.

In illustrative embodiments, the AI layer is disposed directly between the internal backing layer and the release liner. There is not a “reservoir” or pre-formed pocket, as such; rather, the AI layer and internal backing layer are hermetically sealed between the overlay and the release liner.

With such polymeric matrix, the active ingredient does not need to be contained, e.g., in microcapsules or other containment/release means.

In illustrative embodiments, the entire patch is flexible so that it will adhere effectively and comfortably to the contours of the site of application and so that it will withstand the flexions associated with normal living activities.

In illustrative embodiments, the invention is used to contain a volatile component of the AI-containing patch. By “volatile,” is meant that the agent has a vapor pressure above 0.1 mm Hg at 20° C. Volatile components can be, for example, DMSO, a lower (C₁-C₄) alkyl ester of lactic acid such as ethyl lactate and other volatile organic solvents, for example, sulfoxides such as decyl methyl sulfoxide; alcohols such as ethanol, propanols, hexanols, and benzyl alcohol, fatty acids such as valeric acid, isovaleric acid, isopropyl butyrate, ethyl acetate, and butyl acetate; polyols such as butanediol and ethylene glycol; amides such as dimethylacetamide, diethyl toluamide, dimethylformamide, pyrrolidone, and methylpyrrolidone; terpenes such as limonene, pinene, terpinone, mentone, eucalyptus, and menthol; alkanes such as hexane and heptane, and organic acids such as citric acid.

The shape of the device of the invention is not critical. For example, it can be circular, i.e., a disc, or it can be polygonal, e.g., rectangular, or elliptical. The surface area of the AI-containing layer generally does not exceed about 60 cm² in area. It can be, for example, about 5 to 50 cm², including about 8 to about 40 cm² and about 10 to about 20 cm².

FIGS. 1, 2, and 3 illustrate devices comprising an internal backing layer (4), which can in some embodiments inhibit absorption of components of the AI-containing layer into the overlay. Such internal backing layer can be made of any suitable material that is impermeable or substantially impermeable to the AI and to excipients of the adhesive polymer matrix. The internal backing layer serves as a protective cover for the AI-containing layer and provides a support function. The backing layer can be formed so that it is essentially the same size as the hormone-containing adhesive polymer matrix or it can be of larger dimension so that it can extend beyond the edges of the API-containing patch outwardly. The backing layer can be any appropriate thickness that will provide the desired protective and support functions. A suitable thickness is from about 10 to about 300 microns. More specifically, the thickness is less than about 150 microns, yet more specifically, it is less than about 100 microns, and most specifically, the thickness is less than about 50 microns.

Examples of materials suitable for making the internal backing layer are films of polypropylene, polyesters such as poly(ethylene terephthalate), metal foils, metal foil laminates of such suitable polymer films, and the like. If the function of the internal backing layer is combined with the function of the weldable barrier layer, then a single backing layer will perform both functions. In this case, the material used must be ultrasonically weldable to the release liner.

The internal backing layer is, in general, a separate layer from the overlay or any component layer of the overlay, e.g., it is not co-extruded or co-molded with the overlay. In illustrative embodiments, the internal backing layer can be coated on the surface adjacent the AI layer with a “tie-coat,” e.g., a polyvinyl acetate-polyethylene vinyl acetate copolymer or other soft polymer or copolymer.

In illustrative embodiments of the invention, the backing layer that is welded to the release liner (whether it is a separate weldable barrier layer as in FIGS. 1, 3, and 4, the same as the internal backing layer as in FIG. 2, or the same as the overlay as described above) and the release liner have high glass transition temperatures, thereby avoiding softer polymers, such as ethylene vinyl acetate (EVA), often used in heat sealing. In any event weldable layers can be prepared from polymers that are highly crystalline (i.e., >50% of the polymers are in crystalline form and/or that have a high glass transition temperatures (i.e., >about 100° C.)). Use of such harder polymers can result in decreased loss of volatile components. In such embodiments, useful polymers include polyester films, such as Mylar® (DuPont Teijin) and Scotchpak® 9732 (3M Company) and polyacrylonitrile films such as Barex® polyacrylonitrile-methyl acrylate copolymer. In illustrative embodiments, because the backing layer is directly ultrasonically welded to the release liner, there is no need for a separate heat seal between the backing layer and the release liner.

The release liner is made of any material (1) that is impermeable or substantially impermeable to the components of the AI-containing layer, (2) which can form an ultrasonic weld with the weldable backing layer, and (3) that is readily removable by peeling from the API-containing layer just prior to applying to the skin. “Impermeable” and “substantially impermeable,” will be understood to mean that the components of the AI layer, in particular, the volatile components, do not become absorbed by or otherwise pass into or through the release liner such as to alter the performance of the device, in particular, the skin permeability or efficacy of the active ingredients.

The release liner can have the same dimensions as the overlay, discussed below, or it can extend totally or partially beyond the edge of the patch. In one illustrative embodiment, the release liner extends partially beyond the overlay so as to form “tabs” of release liner material that extend beyond the edges of the overlay for easy separation of the release liner from the rest of the system.

Preferably, it comprises a fluorinated or siliconized polyester film. A preferred material for the release liner when the overlay comprises a PIB PSA is a Scotchpak® liner, such as Scotchpak® 1022 or Scotchpak® 9744 fluorinated polyester release liners.

In this illustrative embodiment, a drug-permeable membrane, rate-control membrane, porous membrane, seal peel, peelable disk or other layer, covering or coating between the polymeric matrix and the release liner is not required. Instead, in illustrative embodiments, owing to the viscosity of the polymeric matrix, the release liner is in direct contact with the AI layer and, outside the perimeter of the AI layer, with the overlay.

An illustrative embodiment of the invention comprises an overlay, which, as explained above, may or may not serve as the backing layer that is welded to the release liner. Such overlay can comprise a PSA in which the solubility of the volatile components is less, preferably significantly less, than the solubility of those same components in the AI matrix. So, e.g., when the volatile component is DMSO or ethyl lactate, a PIB PSA may be chosen. Generally, such PIB PSA comprises a mix of a low to medium molecular weight and a high molecular weight PIB, a plasticizer such as polybutene, and a hydrocolloid such as a cross-linked polyvinylpyrrolidine. Useful PIBs include, e.g., Oppanol® PIBs (BASF), which have average molecular weights of between 40,000 and 4,000,000.

Other rubber-based polymers that can be used in place of PIB PSA in the overlay include silicone-based PSAs, such as BIO-PSA® (Dow Corning); copolymers and terpolymers of styrene/butadiene/styrene, styrene/isoprene/styrene, and styrene-ethylene/butylenes-styrene, such as Kraton D styrene/butadiene and Kraton G styrene-ethylene/butylene-styrene or styrene-ethylene/propylene-styrene. Isoprene rubbers, such as Kraton IR linear polyisoprene homopolymers, can also be used.

As shown in FIG. 1, and like the release liner, the overlay (1) can extend beyond the perimeter of the AI-containing layer in all directions, typically by a margin of about 0.1 to about 2 cm, more specifically about 0.5 to about 1.5 cm, and yet more specifically about 0.3 to about 1.2 cm beyond the perimeter of the AI-containing layer.

The overlay, if it comprises a PSA layer, improves adherence to the skin by supplementing the adhesion provided by the PSA in the AI-containing layer, if present, or, in the case of an AI-containing layer that does not comprise a PSA, it provides adherence to the skin.

In addition, in one illustrative embodiment of the invention, the overlay adheres to the release liner around the perimeter of both layers, thereby sealing in the components of the AI-containing layer. This seal is additional to the ultrasonically welded seal.

In another embodiment, there is no PSA layer adjoining the overlay to the release liner, in which case the ultrasonically welded seal is the only seal.

The seal between the overlay PSA and the release liner prevents, or substantially prevents, loss of the components of the AI-containing layer through the seal between these two layers such as during storage of the system.

The overlay can also comprise a covering that does not comprise a PSA, i.e., that comprises a non-PSA layer, such that the surface of the overlay that is exposed to fingers, clothing and ambient dirt or dust is non-tacky, is flexible or malleable so as to flex with skin and muscle movements, is of an unnoticeable or attractive color and texture, and permits moisture from the skin to pass through the device owing to its being porous or otherwise permeable to water.

Thus, it may be desirable to utilize a multi-layered overlay comprising a first layer of a PSA in which the volatile component is insoluble covered with an overlay covering having the properties described above.

While a PIB PSA is useful for containing DMSO or ethyl lactate, or both, in the AI-containing layer, thus providing a second level of protection over and above that provided by the ultrasonically welded layers, the PIB PSA may flow through most overlay coverings having the properties describe above. Such flow of the PIB PSA can cause the device to become tacky and discolored. Therefore, it may be desirable to use an overlay covering that itself comprises two layers, one of which is a polymeric layer interposed between the PIB PSA (an intermediate layer) and a backing layer. Such intermediate layer can be a polyacrylate PSA as described above, because such PSA will substantially prevent flow of the PIB PSA into and through the overlay covering but will substantially not itself migrate into or through the overlay covering.

Thus, in an illustrative embodiment of the invention, the AI-containing layer comprises a polyacrylate matrix further comprising a humectant, e.g., PVP/VA, and skin permeation enhancers including DMSO, ethyl lactate, or both, or another one or more volatile organic solvents; the overlay is a laminate that comprises three layers: a PIB PSA layer, an intermediate layer that comprises a material that does not permit flow of the PIB PSA but that does permit passage of moisture; and an overlay covering (or backing layer) that is non-tacky, attractive, flexible, and moisture permeable.

Materials useful in the intermediate layer include, e.g., polyacrylates, polyurethanes, plasticized polyvinyl chlorides, and copolymers of polyethylene and ethyl vinyl acetate. Rubber-based polymers that are of very high molecular weight, e.g., at least about 150,000 Daltons can also be used, as can rubber-based polymers that can be crosslinked. Examples include the Kraton D styrene/butadiene, Kraton G styrene-ethylene/butylene-styrene or styrene-ethylene/propylene-styrene and Kraton IR linear polyisoprene homopolymers Butyl rubbers and silicone rubbers, which are cross-linkable, can also be used. The intermediate layer can comprise a PSA that binds the first overlay layer as well as the overlay covering. High molecular weight, cross-linked polymers are preferred. Preferably, such PSA is a polyacrylate such as is described above with reference to the AI-containing layer.

Materials used in the overlay covering are generally not PSAs. They include, for example, a polyurethane film, foam or spunbonded structure, a polyolefin foam, a PVC foam or a woven or non-woven fabric. Illustrative wovens include KOB 051, 053 and 055 woven polyesters (Karl Otto Braun.) Illustrative non-woven fabrics include polyesters. An illustrative polyurethane material is CoTran™ 9700 melt-blown polyurethane nonwoven backing (3M), which can be colored in skin tones. Suitable materials are described, e.g., as backing layers in U.S. Pat. No. 6,660,295.

If the overlay covering is not porous, then it can be used without an intermediate layer. However, if the overlay covering is not porous, adhesion problems can result from a build up of moisture in the AI-containing layer and in the overlay. Use of a solid material, i.e., one that is not porous, but that is otherwise permeable to water, such as a thin, e.g., 1 mil (i.e., 0.001 inch), polyurethane film, can be used. However, a porous material such as a foam will, in general, better retain its shape.

### EXAMPLES

The following examples are set forth to describe the invention in greater detail. They are intended to illustrate, not to limit, the invention.

### Example 1

### Welding Feasibility Studies

Welding feasibility studies were performed using a Branson ultrasonic welder. The equipment and set up parameters used to produce samples are given in Table 1.

TABLE 1
Ultrasonic Equipment and Set-up Parameters

| ACTUATOR: | 2000Xaed | DOWN SPEED: | 1.0 IPS |
|---|---|---|---|
| POWER SUPPLY: | 2000Xd, 40 Khz | WELD MODE: | Energy 55 j & 100 j |
| ACCESSORIES: | None | AMPLITUDE: | 90% |
| BOOSTER: | Solver 2:1 Ratio (X2) | HOLD TIME: | 1.0 Seconds |
| PRESSURE: | 50 PSIG 7 60 PSIG | DYNAMIC TRIG.: | 15 lb & 30 lb |
| LOADING: | 40% | | |

ANVIL: Flat faced ring; Medium female knurled plate with urethane pad below
HORN: 40 Khz, 0.87 OD × 0.62 ID w/ 0.04 thick × 0.06 tall ring; 0.87 OD × 0.62 ID w/ female knurled face

Mylar sheet laminates as shown in Layer 3 and Layer 6 in FIG. 1 were the test samples. Layer 3 is a laminate of polyester (Mylar) with a thin tie coat of ethylene vinyl acetate (EVA). Layer 6 is a laminate of polyester (Mylar) with a thin coating of silicone. During the welding Layers 3 and 6 were placed in a way that the silicone coating was in contact with the EVA tie coat.

A prototype sealing anvil was used and several tooling variations were tried, including contacting the top of each sheet with the horn, narrow seal rings versus wider knurled seals and different horn/anvil configurations.

It was determined that strong structural and hermetic welds were obtained which could be peeled off.

### Example 2

### Weld Optimizations

Several pairs of polymer laminates were tested as shown in Table 2. In this table the Barrier Materials are the laminates represented by Layer 3 and Release Liner materials are the laminates represented by Layer 6.

TABLE 2
Materials Used in the Ultrasonically Welding Trial

| | | | | Sample |
|---|---|---|---|---|
| Component | Description | Manufacturer | Mfg designation | Marking |
| Release liner | Silicone treated | Loparex | Polyester 30 Grade | P30 |
| | polyester release liner | | 10393 | |
| | Silicone treated | 3M | 9744 | 9744 |
| | polyester release liner | | | |
| Barrier | Mylar/EVA film (patch | 3M | 9732 | 9732 |
| materials | backing) | | | |
| | Mylar/sealant peal/seal | Perfecseal | 31868G | 31868 |
| | Mylar/sealant peal/seal | DuPont | OL12AF/150 | OL12 |

All pairs formed ultrasonic welds that were peelable. Examination of the weld line before and after peeling apart the two layers revealed that there was movement of materials away from the weld line. It is not clear if the total amounts of the softer polymers, EVA and silicone, were removed from the weld line, but at least enough was removed to from a good seal that was conveniently manually peelable.

### Example 3

### Packaging of Active Patches Within Ultrasonically Welded Mylar Laminates

Transdermal patches containing such highly volatile excipients as DMSO, Ethyl Lactate, Capric Acid and Lauryl Lactate, were sandwiched between the two mylar laminates (3M 9732/Loparex P 30) and then the two laminates were welded ultrasonically. There were two configurations used (Set #1 and Set #2), which were identical to the ones shown in FIGS. 1 and 2. In effect in Set #1, the release liner was welded to the barrier material, outside the perimeter of the active patch; in Set #2, the weld took place inside the perimeter of the active patch, thus creating the weld through the acrylic adhesive patch.

The equipment and set-up parameters were as follows.

EQUIPMENT AND TOOLING SET-UP (SET 1):

| | | | |
|---|---|---|---|
| ACTUATOR: | Ae | DOWN SPEED: | 5% |
| POWER SUPPLY: | 2200 W, 20 Khz | WELD MODE: | Energy 350 j |
| ACCESSORIES: | None | AMPLITUDE: | 100% |
| BOOSTER: | Black 2.5:1 Ratio | HOLD TIME: | 1.0 Seconds |
| PRESSURE: | 32 PSIG | PRE TRIG.: | On, 100% |
| Trigger setting: | Pos 1 | Afterburst | 0.5 s/1.0 s/ |
| | | | 100% |

EQUIPMENT AND TOOLING SET-UP (SET 2):

| | | | |
|---|---|---|---|
| ACTUATOR: | Ae | DOWN SPEED: | 8% |
| POWER SUPPLY: | 2200 W, 20 Khz | WELD MODE: | Energy 100 j |
| ACCESSORIES: | None | AMPLITUDE: | 50% |
| BOOSTER: | Black 2.5:1 Ratio | HOLD TIME: | 1.0 Seconds |
| PRESSURE: | 22 PSIG | PRE TRIG.: | On, 100% |
| Trigger setting: | Pos 1 | Afterburst | 0.5 s/1.0 s/ |
| | | | 100% |

The packaging assemblies were visually evaluated and the peelability of the seals determined to be excellent.

### Example 4

### Loss on Drying (LOD) of Active Excipients at High Temperatures

Samples of the packaged patches from both configurations mentioned above were placed in a 200° C. oven for two days. The loss on drying was determined by weighing the packaged patches before and after drying. Thereafter, the peelable seal was broken and the patches were again exposed to drying, under the same conditions mentioned above. The loss on drying was again determined by weighing the patches before and after drying. The results were as follows:

### Set #1.

LOD while weld was intact: 0.9%
LOD after breaking the weld: 17.4%

### Set #2.

LOD while weld was intact: 1.1%
LOD after breaking the weld: 17.4%

It is obvious from the above data that these ultrasonic peelable welds are hermetic and can be used to control the release of volatile substances in transdermal, topical and cosmetic products.

As illustrated in the Examples, in certain embodiments of the invention the backing layer and the release liner are both polyester films and the ultrasonic weld is formed by applying a weld energy of about 50 to about 500 joules for about 0.25 to about 2 seconds or about 100 to about 350 joules for about 0.5 to about 1.5 seconds, in either case, while the backing layer and release liner are positioned on an anvil.

The present invention is not limited to the embodiments described and exemplified above, but is capable of variation and modification within the scope of the appended claims. Published patent applications and patents referenced in this specification are incorporated herein by reference as though fully set forth.

1. A method of sealing an active ingredient (AI) layer between a backing layer and a release liner in an AI dermal delivery device to be applied to the surface of the skin of a person that comprises ultrasonically welding a seal between the backing layer and the release liner proximate to the perimeter of the AI layer.

2. The method of claim 1 wherein the weld line is within the perimeter of the AI layer.

3. The method of claim 1 wherein the weld line is outside the perimeter of the AI layer.

4. The method of claim 1 wherein the perimeter of the backing layer extends beyond the perimeter of the AI layer, wherein the weld line is outside the perimeter of the AI layer, and there is a PSA layer on the backing that provides adherence of the backing layer to the release liner and the weld is formed through the PSA layer.

5. The method of claim 1 wherein the seal can be broken by manually pulling the release liner away from the backing layer.

6. The method of claim 1 wherein there is a cut through the backing layer such that when the release liner is pulled from the backing layer, the portion of the backing layer on and outside of the weld line remains adhered to the release liner and is removed from the device with the release liner.

7. The method of claim 1 wherein the AI layer comprises one or more volatile components and the backing layer, release liner, and the weld seal there between substantially prevent loss of the volatile component.

8. The method of claim 1 wherein the backing layer, and the weld line, extend beyond the perimeter of the AI layer and wherein there is an internal backing layer between the AI layer and the backing layer, the perimeter of the Internal backing layer being within the weld line.

9. The method of claim 1 wherein there is an Internal backing layer between the AI layer and the backing layer and wherein there is a tie coat between the AI layer and the Internal backing layer for adhering the AI layer to the Internal backing layer.

10. The method of claim 1 wherein the backing layer and the release liner both comprise polymeric films or laminates.

11. The method of claim 10 wherein the polymeric films or laminates, are impermeable to the components of the AI layer.

12. The method of claim 11 wherein the backing layer comprises a polymeric film selected from the group consisting of a polyester film, a polyacrylonitrile film, a polypropylene film and a laminate of two or more of such films.

13. The method of claim 11 wherein the release liner comprises a polymeric film selected from the group consisting of a polyester film, a polyacrylonitrile film, a polypropylene film and a laminate of two or more of such films.

14. The method of claim 13 wherein the release liner is coated with silicone or a fluoropolymer release coating and the Internal backing layer comprises a tie coating for adhering the Internal backing layer to the AI layer.

15. The method of claim 1 wherein the backing layer, and the weld line, extend beyond the perimeter of the AI layer and wherein there is an Internal backing layer between the AI layer and the backing layer, the perimeter of the backing layer being within the weld line, such that the seal between the backing layer and the release liner is not peelable, such device further comprising a kiss cut or through cut in the backing layer within the perimeter of the weld line.

16. The method of claim 1 wherein the device comprises a through cut in the backing layer within the perimeter of the weld line, and such through cut is a perforated through cut.

17. The method of claim 1 wherein the device comprises an overlay.

18. The method of claim 17 wherein the overlay is adhered to the backing layer by a pressure sensitive adhesive.

19. The method of claim 18 wherein the overlay comprises a PSA layer that is adhered to the release liner outside the perimeter of the AI layer.

20. The method of claim 1 wherein the AI layer comprises a PSA and one or more volatile components.

21. The method of claim 1 wherein the AI layer comprises DMSO or ethyl lactate or both.

22. A dermal delivery device for adherence to surface of the skin of a person that comprises an AI layer, a backing layer, and a release liner, having an ultrasonically welded seal between the backing layer and the release liner.

23. A dermal delivery device for adherence to surface of the skin of a person that comprises an AI layer, a backing layer, and a release liner, having an ultrasonically welded seal between the backing layer and the release liner, and having been prepared by the method of claim 19.
